(19) United States
(12) Patent Application Publication
(10) Pub. No.: US 2003/0236237 A1
(43) Pub. Date: Dec. 25, 2003

(54) Method of treating symptoms of hormonal variation, including hot flashes, using tachykinin receptor antagonist

(76) Inventor: Thomas J. Guttuso JR., Rochester, NY (US)

Correspondence Address: Nixon Peabody LLP, Clinton Square, P.O. Box 31051, Rochester, NY, 14603-1051, US

(21) Appl. No.: 10/609,176

(22) Filed: Jun. 27, 2003

Related U.S. Application Data
(63) Continuation of application No. 09/879,390, filed on Jun. 12, 2001.
(60) Provisional application No. 60/211,116, filed on Jun. 12, 2000.

(51) Int. Cl. A61K 31/56

(52) U.S. Cl. 514/171

(57) ABSTRACT

The present invention relates to a methods of treating hot flashes and symptoms of hormonal variation in a patient, which methods include providing a tachykinin receptor antagonist and administering the tachykinin receptor antagonist to a patient experiencing a symptom of hormonal variation under conditions effective to treat the symptom of hormonal variation, which symptoms of hormonal variation can include hot flashes.

This application is a continuation of U.S. patent application Ser. No. 09/879,390, filed Jun. 12, 2001, which claims priority benefit of U.S. Provisional Patent Application Serial No. 60/211,116, filed Jun. 12, 2000, each of which is hereby incorporated by reference in its entirety.

### FIELD OF THE INVENTION

The present invention relates generally to methods of treating symptoms of hormonal variation, including hot flashes.

### BACKGROUND OF THE INVENTION

Hot flashes or flushing occur commonly in menopausal women. This is characterized by a sudden onset of warmth in the face and neck and often progressing to the chest. Such an episode generally lasts several minutes and is evidenced by a visible flushing of the skin. Often such episodes are accompanied by sweating, dizziness, nausea, palpitations and diaphoresis. Such symptoms can disrupt sleep and interfere with the quality of life. Although the cause of hot flashes are not completely understood, they are thought to be a disorder of thermoregulation resulting from a transient lowering of the hypothalamic temperature regulatory set point (Kronenberg et al., “Thermoregulatory Physiology of Menopausal Hot Flashes: A Review,” Can. J. Physiol. Pharmacol., 65:1312-1324 (1987)). In post-menopausal woman, the cause of such hot flashes is believed to be a consequence of declining estrogen levels. Thus, it is not surprising that hot flashes also occur in a high percentage of women taking the anti-estrogen drug tamoxifen.

Men may also have hot flashes following androgen-deprivation therapy (from bilateral orchiectomy or treatment with a gonadotrophin-releasing-hormone agonist) for metastatic prostate cancer.

Although estrogen replacement therapy is the most direct and effective treatment for hot flashes in women, there are women for whom such therapy is contraindicated, i.e., women with breast cancer or a strong family history of breast cancer, a history of clotting, severe migraine, or who are averse to taking the drug.

In these women, there are alternative medications to prevent or treat the serious consequences of menopause, such as osteoporosis and raised serum lipid levels. Included in this category are the selective estrogen-receptor modulators (SERMs), such as raloxifene (see U.S. Pat. No. 5,534,526 to Cullinan), which selectively bind to and activate the estrogen receptors of some tissues such as bone, and block the receptors of others, i.e., breast and uterus. In so doing, they lack the negative impact that prolonged estrogen therapy may have on these organs. However, in contrast to estrogen, SERMs are not as effective in preventing hot flashes.

Other than estrogen-replacement therapy, there are no effective means to alleviate hot flashes. Low dose oral megestrol acetate, a progestational agent, was shown to reduce the frequency of hot flashes in both men and women in a short term study (Loprinzi et al., “Megestrol Acetate for the Prevention of Hot Flashes,” N. Engl. J. Med. 331:347-351 (1994)). However, chronic adrenal insufficiency can be a side effect of low dose megestrol acetate when taken long term. Transdermal clonidine, a centrally active α-agonist, had only a moderate effect on the frequency and severity of hot flashes in tamoxifen-treated women (Goldberg et al., “Transdermal Clonidine for Ameliorating Tamoxifen-induced Hot Flashes,” J. Clin. Onc. 12:155-158 (1994)).

Accordingly, there is a need for an alternative method of treating symptoms of hormonal variation, including hot flashes, which overcomes the deficiencies in the relevant art.

### SUMMARY OF THE INVENTION

A first aspect of the present invention relates to a method of treating hot flashes in a patient which includes providing a tachykinin receptor antagonist and administering the tachykinin receptor antagonist to a patient experiencing hot flashes under conditions effective to treat the hot flashes.

A second aspect of the present invention relates to a method for treating a symptom of hormonal variation in a patient which includes providing a tachykinin receptor antagonist and administering the tachykinin receptor antagonist to a patient experiencing a symptom of hormonal variation under conditions effective to treat the symptom of hormonal variation.

The present invention provides an improved treatment for symptoms of hormonal variation, including hot flashes, which can be significantly uncomfortable and seriously affect one's quality of life. Tachykinin receptor antagonists, which can inhibit the activity of neurokinins on their receptors, can be administered in a manner which is effective to reduce or substantially eliminate the occurrence or severity of hot flashes. Current trials involving the administration of various tachykinin receptor antagonists for other uses have demonstrated that a number of such antagonists are well tolerated by patients.

### DETAILED DESCRIPTION OF THE INVENTION

A first aspect of the present invention relates to a method of treating hot flashes in a patient which includes providing a tachykinin receptor antagonist and administering the tachykinin receptor antagonist to a patient experiencing hot flashes under conditions effective to treat the hot flashes.

A second aspect of the present invention relates to a method for treating a symptom of hormonal variation in a patient which includes providing a tachykinin receptor antagonist and administering the tachykinin receptor antagonist to a patient experiencing a symptom of hormonal variation under conditions effective to treat the symptom of hormonal variation.

Tachykinins are small peptides found in the central and peripheral nervous systems. Three different tachykinins have been identified in mammals: substance P, neurokinin A, and neurokinin B. Each of these acts as a neurotransmitter and neuromodulator (Maggi et al., “Tachykinin and Tachykinin Receptors,” J. Auton. Pharmacol. 13:23-93 (1993); Nakanishi, “Mammalian Tachykinin Receptors,” Ann. Rev. Neurosci. 14:123-136 (1991), which are hereby incorporated by reference in their entirety). The diverse effects of these tachykinins are mediated by three receptors: NK₁, NK₂, and NK₃, all of which belong to the superfamily of G-protein coupled receptors (Maggi et al., “Neuropeptides as Regulators of Airway Function: Vasoactive Intestinal Peptide and the Tachykinins,” Physiol. Rev. 151:277-322 (1995); Maggi, “The Mammalian Tachykinin Receptors,” Gen. Pharmacol. 26:911-944 (1995), which are hereby incorporated by reference in their entirety). The NK₁ receptor prefers substance P, the NK₂ receptor prefers neurokinin A, and the NK₃ receptor prefers neurokinin B.

In both the postmenopausal woman and the ovariectomized rat, there is a dramatic increase in neurokinin B gene expression in the hypothalamic arcuate nucleus (ArN)in rat and human there is roughly a 2-fold and 15-fold increase, respectively, in ArN cells expressing NKB, while in both rat and human there is a 2-fold increase in ArN NKB grain density (Rance and Bruce, “Neurokinin B gene expression is increased in the arcuate nucleus of ovariectomized rats,” Neuroendocrinology 60:337-345 (1994); Rance and Young, “Hypertrophy and increased gene expression of neurons containing neurokinin-B and substance-P messenger ribonucleic acids in the hypothalami of postmenopausal women,” Endocrinol. 128:2239-2247 (1991), which are hereby incorporated by reference in their entirety). In humans and rats, the enhanced NKB gene expression is isolated to the ArN. Furthermore, treatment of the ovariectomized rat or primate with estrogen completely prevents these ArN changes (Rance and Bruce, “Neurokinin B gene expression is increased in the arcuate nucleus of ovariectomized rats,” Neuroendocrinology 60:337-345 (1994); Abel et al., “The effects of hormone replacement therapy on hypothalamic neuropeptide gene expression in a primate model of menopause,” J. Clin. Endocrinol. & Metab. 84:2111-2118 (1999), which are hereby incorporated by reference in their entirety). Since estrogen therapy in postmenopausal women is the most effective treatment of hot flashes, estrogen's effects on NKB expression in the ArN may be involved with estrogen's mechanism of action. This is supported by the observation that systemic injection of substance-P, a closely related tachykinin to NKB, results in a clinical hot flash in humans (Schaffalitzky De Muckadell et al., “Flushing and plasma substance P concentration during infusion of synthetic substance P in normal man,” Scand. J. Gastroenterology 21:498-502 (1986), which is hereby incorporated by reference in its entirety).

It has been postulated that hot flashes result from a transient lowering of the hypothalamic temperature regulatory set point (Kronenberg and Downey, “Thermoregulatory physiology of menopausal hot flashes: a review,” Canad. J. Physiol. Pharmacol. 65:1312-1324 (1987), which is hereby incorporated by reference in its entirety). This results in a sudden perception of heat and activation of physiological cooling processes such as sweating and cutaneous vasodilation—i.e., a hot flash. The medial preoptic area (MPOA) is the principle nucleus regulating heat-loss physiology (Sirnerly and Swanson, “Projections of the medial preoptic nucleus: a Phaseolus vulgaris leucoagglutinin anterograde tract-tracing study in the rat,” J. Comp. Neurology 270:209-242 (1988), which is hereby incorporated by reference in its entirety). Direct stimulation of the MPOA results in a transient physiological response that mimics a hot flash—cutaneous vasodilation, sweating, and panting (Day et al., “Thermoregulatory effects of preoptic area injections of noradrenaline in restrained and unrestrained rats,” Brain Res. 174:175-179 (1979); Casper and Yen, “Neuroendocrinology of menopausal flushes: an hypothesis of flush mechanism,” Clin. Endocrinol. 22:293-312 (1985), which are hereby incorporated by reference in their entirety). The ArN has a major projection to the MPOA (Akesson et al., “Estrogen-concentrating hypothalamic and limbic neurons project to the medial preoptic nucleus,” Brain Res. 451:381-385 (1988), which is hereby incorporated by reference in its entirety). The MPOA also receives its largest substance-P input from the ventromedial hypothalamus (VMH) (Yamano et al., “A substance P-containing pathway from the hypothalamic ventromedial nucleus to the medial preoptic area of the rat: an immunohistochemical analysis,” Neuroscience 18:395-402 (1986), which is hereby incorporated by reference in its entirety).

Without being bound by theory, it is believed that postmenopausal (as well as surgically or chemically induced) hot flashes result from overstimulation of the MPOA by tachykinin projections from the ArN and the VMH which are disinhibited by the low estrogen state. The overactivity of the ArN tachykinin cells is mediated by an upregulation of their α₂δ subunits of voltage-gated calcium channels which, in turn, increases membrane calcium permeability causing increased cellular activity and neurotransmitter production. This MPOA overstimulation eventually reaches a threshold when the MPOA activates a lowering of the thermoregulatory set point and, consequently, a hot flash is experienced.

As used in the present invention, the tachykinin receptor antagonist can be an NK₁ receptor antagonist, an NK₂ receptor antagonist, an NK₃ receptor antagonist, or a tachykinin receptor antagonist which has antagonist effects at more than one of the several NK receptors. Preferably, the tachykinin receptor antagonist is an NK₁ receptor antagonist, an NK₃ receptor antagonist, or an antagonist of both the NK₁ receptor and the NK₃ receptor.

In addition, various combinations of NK₁ receptor antagonists, NK₂ receptor antagonists, and NK₃ receptor antagonists can be administered together. For example, such combinations may include an NK₁ receptor antagonist in combination with an NK₂ receptor antagonist, an NK₁ receptor antagonist in combination with an NK₃ receptor antagonist, an NK₂ receptor antagonist in combination with NK₃ receptor antagonist, or an NK₁ receptor antagonist in combination with both an NK₂ receptor antagonist and an NK₃ receptor antagonist. One preferred combination is an NK₁ receptor antagonist together with an NK₃ receptor antagonist.

A number of NK receptor antagonists have been described in the art and can be prepared according to known procedures as identified, for example, in the following references: U.S. Pat. No. 5,344,830 to Mills et al., U.S. Pat. No. 5,554,627 to Lewis et al., U.S. Pat. No. 5,554,641 to Horwell et al., U.S. Pat. No. 5,563,161 to Huscroft et al., U.S. Pat. No. 5,594,022 to Horwell et al., U.S. Pat. No. 5,607,936 to Chiang et al., U.S. Pat. No. 5,610,145 to Horwell et al., U.S. Pat. No. 5,610,165 to MacCoss et al., U.S. Pat. No. 5,612,336 to Lewis et al., U.S. Pat. No. 5,624,947 to Keown et al., U.S. Pat. No. 5,627,211 to Teall et al., U.S. Pat. No. 5,633,266 to Baker et al., U.S. Pat. No. 5,635,509 to Jacobs et al., U.S. Pat. No. 5,633,281 to Teall et al., U.S. Pat. No. 5,654,316 to Carruthers et al., U.S. Pat. No. 5,663,352 to MacLeod et al., U.S. Pat. No. 5,665,883 to Baker et al., U.S. Pat. No. 5,688,960 to Shankar, U.S. Pat. No. 5,696,123 to Dollinger et al., U.S. Pat. No. 5,696,267 to Reichard et al., U.S. Pat. No. 5,698,710 to Sisto et al., U.S. Pat. No. 5,708,006 to Dollinger et al., U.S. Pat. No. 5,719,156 to Shue et al., U.S. Pat. No. 5,731,309 to Bernstein et al., U.S. Pat. No. 5,760,018 to Baker et al., U.S. Pat. No. 5,760,248 to Sisto et al., U.S. Pat. No. 5,846,965 to MacKenzie et al., U.S. Pat. No. 5,849,795 to Sisto et al., U.S. Pat. No. 5,892,039 to Shue et al., U.S. Pat. No. 5,919,803 to Giblin et al., U.S. Pat. No. 5,922,744 to Harrison et al., U.S. Pat. No. 5,935,972 to Naylor et al., U.S. Pat. No. 5,945,428 to Shih et al., U.S. Pat. No. 5,962,485 to Owens, U.S. Pat. No. 5,968,923 to MacKenzie et al., U.S. Pat. No. 5,968,929 to Blythin et al., U.S. Pat. No. 6,013,652 to MacCoss et al., U.S. Pat. No. 6,020,346 to Armour et al., U.S. Pat. No. 6,046,195 to Haworth et al., U.S. Pat. No. 6,060,469 to Baker et al., U.S. Pat. No. 6,063,926 to Reichard et al., U.S. Pat. No. 6,103,719 to Esser et al., U.S. Pat. No. 6,110,919 to Howard et al., U.S. Pat. No. 6,150,325 to Arcamone et al., U.S. Pat. No. 6,204,265 to Reichard et al., U.S. Pat. No. 6,207,678 to Monaghan et al., U.S. Pat. No. 6,242,438 to MacKenzie et al., and U.S. Pat. No. 6,235,732 to Dollinger et al., Wallace et al., “A double ring closing metathesis reaction in the rapid, enantioselective synthesis of NK-1 receptor antagonists,” Org. Lett. 3(5):671-674 (2001), Reichard et al., “The design and synthesis of novel NK1/NK2 dual antagonists,” Bioorg. Med. Chem. Lett. 10(20):2329-2332 (2000), Liu et al., “Synthesis of a substance P antagonist with a somatostatin scaffold: factors affecting agonism/antagonism at GPCRs and the role of pseudosymnuetry,” J. Med. Chem. 43(21):3827-3831 (2000), Nishi et al., “Combined tachykinin receptor antagonist: synthesis and stereochemical structure-activity relationships of novel morpholine analogues,” Bioorg. Med. Chem. Lett. 10(15):1665-668 (2000), Rosen et al., “Synthesis and structure-activity relationships of CP-122,721, a second-generation NK-1 receptor antagonist,” Bioorg. Med. Chem. Lett. 8(3):281-284 (1998), Caliendo et al., “Synthesis and in vitro activities of NK-1 antagonists derived from L-tryptophan,” Farmaco 52(10):589-593 (1997), Kubota et al., “Spiro-substituted piperidines as neurokinin receptor antagonists: Design and synthesis of (+/−)-N-[2(3,4-dichlorophenyl)-4-(spiro [isobenzo furan-1(3H),4′piperidin]-1′-yl)butyl]-N-methylbenzamide, YM-35375, as a new lead compound for novel neurokinin receptor antagonists,” Chem. Pharm. Bull. (Tokyo) 46(2):351-354 (1998), Hirschmann et al., “Synthesis of potent cyclic hexapeptide NK-1 antagonists. Use of a minilibrary in transforming a peptidal somatostatin receptor ligand into an NK-1 receptor ligand via a polyvalent peptidomimetic,” J. Med. Chem. 39(13):2441-2448 (1996), Caliendo et al., “Synthesis and in vitro activities of highly potent and selective tripeptide antagonists of the neurokinin NK-1 receptor,” Farmaco 50(11):755-759 (1995), Karagiannis et al., “Synthesis of a potent antagonist of substance P by replacing the CH₂SCH₃ and the alpha-carboxamide groups of the methionine at [Om6]-SP6-11 by benzyl ester groups,” Int. J. Pept. Protein Res. 42(6):565-569 (1993), Rosen et al., “Synthesis, in vitro binding profile, and autoradiographic analysis of [3H]-cis-3-[(2-methoxybenzyl)amino]-2-phenylpi peridine, a highly potent and selective nonpeptide substance P receptor antagonist radioligand,” J. Med. Chem. 36(21):3197-3201 (1993), Caliendo et al., “Synthesis and neurokinin antagonist activity of 2-benzylidene- and 2-benzyl-3-benzylamino quinuclidines,” Farmaco 48(10):1359-1378 (1993), Manolopoulou et al., “Synthesis of potent antagonists of substance P by modifying the methionyl and glutaminyl residues of its C-terminal hexapeptide and without using D-amino acids,” Int. J. Pept. Protein Res. 41(4):411-414 (1993), Lawrence et al., “Synthesis and substance P antagonist activity of naphthimidazolium derivatives,” J. Med. Chem. 35(7):1273-1279 (1992), Aitken et al., “Synthesis, modeling and NK1 antagonist evaluation of a non-rigid cyclopropane-containing analogue of CP-99,994,” Bioorg. Med. Chem. Lett. 11(5):659-661 (2001), Reichard et al., “The design and synthesis of novel NK1/NK2 dual antagonists,” Bioorg. Med. Chem. Lett. 10(20):2329-2332 (2000), Elling et al., “Disulfide bridge engineering in the tachykinin NK1 receptor,” Biochemistry 39(4):667-675 (2000), all of which are hereby incorporated by reference in their entirety. Other NK receptor antagonists, now known or hereafter developed, can also be used according to the present invention.

A number of suitable NK₁ receptor antagonists are currently in clinical trials for other indications. These include, without limitation: GR 203040 which is available from GlaxoSmithKliine (Research Triangle Park, N.C.); CP 99994 which is available from Pfizer (Groton, Conn.); CP 122721 (Rosen et al., “Synthesis and structure-activity relationships of CP-122,721, a second-generation NK-1 receptor antagonist,” Bioorg. Med. Chem. Lett. 8(3):281-284 (1998), which is hereby incorporated by reference in its entirety) which is available from Pfizer; GR 205171 which is available from GlaxoSmithKline; PD 154075 which is available from Pfizer (Parke-Davis); FK 888 which is available from Fujisawa Healthcare, Inc. (Deerfield, Ill.); RP 67580 which is available from Aventis (Strasbourg, France); L 760735 which is available from Merck (Whitehouse Station, N.J.); TAK 637 which is available from Takeda Pharmaceuticals N.A. (Lincolnshire, Ill.); R116301 which is available from Cinalfa AG (Läufelfingen, Switzerland); RPR 100893 or dapitant (CAS 153438-49-4), which is available from Aventis; L 754030 and its prodrug L 758298, both available from Merck; MK 869 which is available from Merck; SR 140333, which is available from Sanofi-Synthelabo (Malvern, Pa.); NKP 608, which is available from Novartis (East Hanover, N.J.); GR 73632 which is available from GlaxoSmithKliine; MEN 11467 which is available from Menarini Group (Firenze, Italy), as well as pharmaceutically acceptable salts thereof. Suitable salts can be prepared according to known techniques. Combinations of one or more NK₁ receptor antagonists can also be administered.

A number of suitable NK₂ receptor antagonists are currently in clinical trials for other indications. These include, without limitation: SR 48968 or saredutant (CAS No. 142001-63-6), which is available from Sanofi-Synthelabo; and MEN 10627 (Quartara et al., “A review of the design, synthesis and biological activity of the bicyclic hexapeptide tachykinin NK2 antagonist MEN 10627,” Regul. Pept. 65(1):55-59 (1996); Caciagli et al., “Large-scale production of peptides using the solid-phase continuous flow method. Preparative synthesis of the novel tachykinin antagonist MEN 10627,” J. Pept. Sci. 3(3):224-230 (1997), which are hereby incorporated by reference in their entirety), which is available from Menarini Group, as well as pharmaceutically acceptable salts thereof. Suitable salts can be prepared according to known techniques. Combinations of one or more NK₂ receptor antagonists can also be administered.

A number of suitable NK₃ receptor antagonists are currently in clinical trials for other indications. These include, without limitation: SB-223412-A or talnetant hydrochloride (CAS 204519-66-4) which is available from GlaxoSmithKline, and SR 142801 or osanetant (CAS 160492-56-8) which is available from Sanofi-Synthelabo, as well as pharmaceutically acceptable salts thereof. Suitable salts can be prepared according to known techniques. Combinations of one or more NK₃ receptor antagonists can also be administered.

The present invention requires administration of the tachykinin receptor antagonist under conditions effective to treat either a symptom of hormonal variation or, more specifically, hot flashes (whether hormonally, surgically, drug, or otherwise induced). The effective conditions typically involve administering an amount of such compound that is effective for the desired treatment. By treating the symptom of hormonal variation, including hot flashes, the present invention encompasses either reducing the number of symptomatic events, reducing the severity of symptomatic events, or both.

Effective amounts of the tachykinin receptor antagonist will depend upon the mode of administration, frequency of administration, and the type of pharmaceutical composition used to deliver the compound into a patient. Generally, effective amounts of such compounds will be about 0.01 to about 300 mg/kg·body wt. per day, preferably about 0.1 to about 200 mg/kg·body wt. per day, more preferably about 1 to about 100 mg/kg·body wt. per day. Typical daily doses will be from about 10 to about 5000 mg per day for an average adult patient of normal weight. While individual needs vary, determination of optimal ranges of effective amounts of each compound is within the skill of the art. For tachykinin receptor antagonists which are involved in clinical trials for other indications, the safe and effective dosages identified in such trials can be considered when selecting dosages for treatments according to the present invention.

The tachykinin receptor antagonists used according to the present invention can be administered alone or as a pharmaceutical composition, which includes the compound(s) and a pharmaceutically-acceptable carrier. In forms available from the above-listed manufacturers, the tachykinin receptor antagonists are typically provided as a pharmaceutical composition.

The pharmaceutical composition can also include suitable excipients, or stabilizers, and can be in solid or liquid form such as, tablets, capsules, powders, solutions, suspensions, or emulsions. Typically, the composition will contain from about 0.01 to 99 percent, preferably from about 5 to 95 percent of active compound(s), together with the carrier.

The tachykinin receptor antagonist, when combined with pharmaceutically or physiologically acceptable carriers, excipients, or stabilizers, whether in solid or liquid form such as, tablets, capsules, powders, solutions, suspensions, or emulsions, can be administered orally, parenterally, subcutaneously, intravenously, intramuscularly, intraperitoneally, by intranasal instillation, by implantation, by intracavitary or intravesical instillation, intraocularly, intraarterially, intralesionally, transdermally, or by application to mucous membranes, such as, that of the nose, throat, and bronchial tubes (i.e., inhalation).

For most therapeutic purposes, the tachykinin receptor antagonist can be administered orally as a solid or as a solution or suspension in liquid form, via injection as a solution or suspension in liquid form, or via inhalation of a nebulized solution or suspension.

The solid unit dosage forms can be of the conventional type. The solid form can be a capsule, such as an ordinary gelatin type containing the compounds of the present invention and a carrier, for example, lubricants and inert fillers such as, lactose, sucrose, or cornstarch. In another embodiment, these compounds are tableted with conventional tablet bases such as lactose, sucrose, or cornstarch in combination with binders like acacia, cornstarch, or gelatin, disintegrating agents, such as cornstarch, potato starch, or alginic acid, and a lubricant, like stearic acid or magnesium stearate.

For injectable dosages, solutions or suspensions of these materials can be prepared in a physiologically acceptable diluent with a pharmaceutical carrier. Such carriers include sterile liquids, such as water and oils, with or without the addition of a surfactant and other pharmaceutically and physiologically acceptable carrier, including adjuvants, excipients or stabilizers. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions.

For use as aerosols, the compound in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

For transdermal routes, the compound is present in a carrier which forms a composition in the form of a cream, lotion, solution, and/or emulsion. The composition can be included in a transdermal patch of the matrix or reservoir type as are conventional in the art for this purpose.

It is also contemplated that administration of the tachykinin receptor antagonist can be carried out in combination with other suitable therapeutic treatments which are useful for treating symptoms of hormonal variation, including hot flashes.

The patient to be treated is any mammalian patient, preferably a human patient. The patient can be either a female patient or a male patient, although the ultimate cause of hot flashes can, of course, be markedly different for both groups of patients. For example, in female patients the hot flash is a primary symptom resulting from menopausal or postmenopausal hormonal variation. However, the hot flash can also be drug-induced by anti-estrogen compounds (e.g., tamoxifen, leuprolide acetate, etc.) or surgically-induced by removal of estrogen-producing tissues (e.g., total abdominal hysterectomy, bilateral salpingo-oophorectomy, etc.). In male patients, the hot flashes typically occur as a side-effect of androgen-dependent therapy for metastatic prostate cancer. They can be either surgically-induced (e.g., bilateral orchiectomy) or drug-induced (e.g., treatment with a gonadotrophin-releasing-hormone agonist, leuprolide acetate, etc.).

### EXAMPLES

The following examples are provided to illustrate embodiments of the present invention but are by no means intended to limit its scope.

### Example 1

### Effect of NK₃ Receptor Antagonist on Controlling Hot Flashes in Postmenopausal Women

Postmenopausal women who report seven (7) or more hot flashes a day will participate in the study. For each patient, serum follicle stimulating hormone and estradiol levels will need to be in the postmenopausal range.

Talnetant hydrochloride (GlaxoSmithKline) is to be administered as an NK₃ receptor antagonist. Patients will be randomly assigned to talnetant hydrochloride treatment or placebo treatment using a double-blind protocol. Dosing of the talnetant hydrochloride will correspond to that which is found to be safe and appropriate in phase 1-3 clinical trials for other demonstrated uses. Low, medium, and high therapeutic dosing of talnetant hydrochloride will be compared against placebo treatment.

To monitor efficacy of placebo and medication, patients will record the frequency and severity of hot flashes in a daily diary. Data from the hot flash daily diaries will be analyzed by ANOVA for statistical significance, considering the talnetant hydrochloride dosage.

### Example 2

### Effect of NK₁ Receptor Antagonist on Controlling Hot Flashes in Postmenopausal Women

Postmenopausal women who report seven (7) or more hot flashes a day will participate in the study. For each patient, serum follicle stimulating hormone and estradiol levels will need to be in the postmenopausal range.

Dapitant (Aventis) is to be administered as an NK₁ receptor antagonist. Patients will be randomly assigned to dapitant treatment or placebo treatment using a double-blind protocol. Dosing of the dapitant will correspond to that which is found to be safe and appropriate in phase 1-3 clinical trials for other demonstrated uses. Low, medium, and high therapeutic dosing of dapitant will be compared against placebo treatment.

To monitor efficacy of placebo and medication, patients will record the frequency and severity of hot flashes in a daily diary. Data from the hot flash daily diaries will be analyzed by ANOVA for statistical significance, considering the dapitant dosage.

Although the invention has been described in detail for the purposes of illustration, it is understood that such detail is solely for that purpose, and variations can be made therein by those skilled in the art without departing from the spirit and scope of the invention which is defined by the following claims.

What is claimed:

1. A method of treating hot flashes in a patient comprising:
providing a tachykinin receptor antagonist selected from the group of NK₂ receptor antagonists and NK₃ receptor antagonist; and
administering the tachykinin receptor antagonist to a patient experiencing hot flashes under conditions effective to treat the hot flashes.

2. The method according to claim 1, wherein the tachykinin receptor antagonist is a NK₂ receptor antagonist.

3. The method according to claim 2, wherein the NK₂ receptor antagonist is selected from the group consisting of saredutant, MEN 10627, and combinations thereof.

4. The method according to claim 1, wherein the tachykinin receptor antagonist is a NK₃ receptor antagonist.

5. The method according to claim 4, wherein the NK₃ receptor antagonist is selected from the group consisting of talnetant hydrochloride, osanetant, and combinations thereof.

6. The method according to claim 1, wherein the tachykinin receptor antagonist comprises a combination of tachykinin receptor antagonists.

7. The method according to claim 6, wherein the combination of tachykinin receptor antagonists comprises a second tachykinin receptor antagonist selected from the group of NK₁ receptor antagonists, NK₂ receptor antagonists, and NK₃ receptor antagonists.

8. The method according to claim 1, wherein the tachykinin receptor antagonist is administered in an amount of about 10 to about 5000 mg per day.

9. The method according to claim 1, wherein the patient is a female patient.

10. The method according to claim 9, wherein the female patient is postmenopausal.

11. The method according to claim 10, wherein menopause is drug induced, surgically induced, or naturally-occurring.

12. The method according to claim 11, wherein menopause is drug induced.

13. The method according to claim 12, wherein the drug is an antiestrogen compound.

14. The method according to claim 13, wherein the anti-estrogen compound is tamoxifen.

15. The method according to claim 1, wherein the patient is a male patient.

16. The method according to claim 15, wherein the male patient experiences drug induced hot flashes.

17. The method according to claim 16, wherein the drug is an antiandrogen compound.

18. The method according to claim 17, wherein the anti-androgen compound is leuprolide acetate.

19. The method according to claim 1, wherein said administration is carried out orally, parenterally, subcutaneously, intravenously, intramuscularly, intraperitoneally, by intranasal instillation, by implantation, by intracavitary or intravesical instillation, intraocularly, intraarterially, intralesionally, transdermally, or by application to mucous membranes.

20. The method according to claim 1, wherein the tachykinin receptor antagonist is present in a pharmaceutical composition comprising the tachykinin receptor antagonist and a pharmaceutically-acceptable carrier.

21. The method according to claim 20, wherein the pharmaceutical composition is in a liquid or solid dosage form.
